(19) United States
(12) Plant Patent Application Publication
(10) Pub. No.: US 2016/0113167 P1
(43) Pub. Date: Apr. 21, 2016

(54) Blueberry plant, EB 9-2

(50) ;

(71) Applicant: Vincent David Mazzardis, Joondalup (AU)

(72) Inventor: Vincent David Mazzardis, Joondalup (AU)

(73) Assignees: Prunus Persica Pty Ltd., Osborne Park (AU); Rolfe Nominees Pty Ltd, Toowoomba (AU)

(21) Appl. No.: 14/756,610

(22) Filed: Sep. 24, 2015

(30) Foreign Application Priority Data
Oct. 16, 2014 (AU) 2014/243

(51) Int. Cl. A01H 5/00

(52) U.S. Cl. PLT/157

(57) ABSTRACT

A new and distinct variety of blueberry plant, which is denominated varietally as ‘EB 9-2’, and which produces a large, to very large fruit, and which further is mature for harvesting and shipment in the early season under the ecological conditions prevailing in Yanchep Springs, Western Australia.

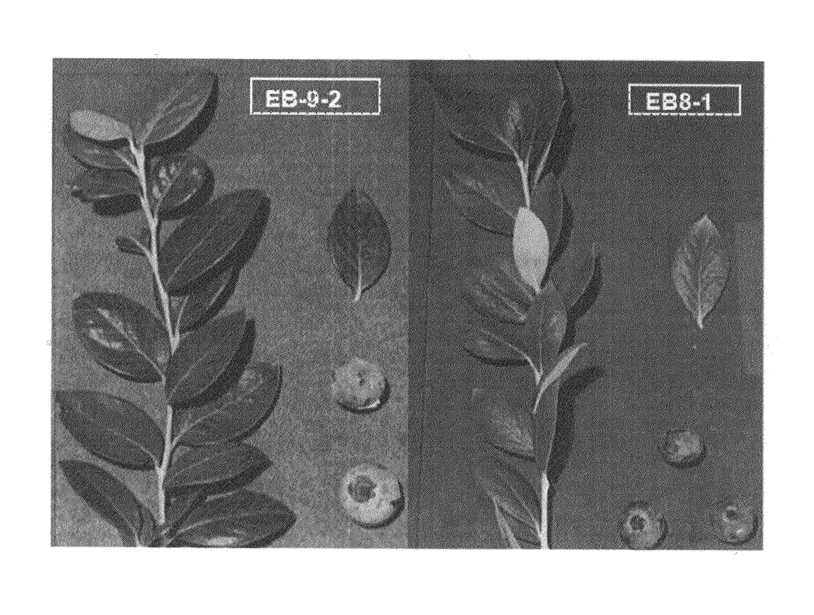

### LATIN NAME

‘Vaccinium Hybrid’

### VARIETAL DENOMINATION

‘EB 9-2’

### RELATED APPLICATION DATA

The present application claims priority to Australian Plant Breeders Rights application Serial No. 2014/243, and which was filed one Oct. 16, 2014, and which was further accepted on Dec. 23, 2014.

### BACKGROUND OF THE NEW VARIETY

The present invention relates to a new, novel, and distinct variety of blueberry plant ‘Vaccinium Hybrid,’ and which has been denominated varietally, hereinafter, as ‘EB 9-2’.

### ORIGIN

The present variety of blueberry plant resulted from an ongoing development program which I have conducted to identify such plants. The purpose of this program is to improve the commercial quality of blueberry plants, and other species. To this end, I have made controlled hybrid cross-pollinations in order to produce plant populations from which improved progeny are evaluated, and thereafter selected.

The blueberry plant ‘EB 9-2’ was originated by me and selected from a population of new plants growing at my farm which is located near Yanchep Springs, in Yanchep, Western Australia. The new variety of blueberry plant was derived from a controlled cross-pollination of the seed parent, blueberry plant ‘BB-1’ [unpatented], and a pollen parent, blueberry plant ‘03-2’ [unpatented], during the 2006 growing season. The seed parent, blueberry parent ‘BB-1’ is principally characterized by a semi-upright bush-type growing habit; a mid-season flowering date; and which further produces medium to large, firm, fruit. Still further, the pollen parent, blueberry plant ‘03-2’ is principally characterized by a semi-upright growth habit; a mid-season flowering date; and which further produces large fruit. Following this cross-pollination, seed from the seed parent ‘BB-1’ produced approximately 500 plants. The first fruiting of these new plants was first observed in 2008. A complete assessment of the fruit, and growth habits of these new plants were evaluated in 2009. After this full assessment, the newly discovered variety ‘EB 9-2’ was selected for further asexual propagation and study. Further, asexual reproduction of the newly discovered variety ‘EB 9-2’ occurred during the 2010 through 2014 growing seasons. The subsequently conducted evaluations of the newly asexually propagated plants, and the fruit produced thereby, has lead the inventor to conclude that the newly discovered blueberry plant variety, ‘EB 9-2’, is a distinct, and new variety in view of its displayed upright, to semi-upright bush-type growth habit; large to very large and oblate shaped fruit; and which further has an excellent flavor; and a very early flowering, and fruit maturity dates in relative comparison to the closest known varieties.

In comparison to the seed parent ‘BB-1’, the present, newly discovery plant is clearly distinguishable therefrom based upon its flowering date. For example, the seed parent flowering date is considered mid-season under the ecological conditions prevailing in Yanchep Springs, Western Australia. On the other hand, the present, new variety has a date of flowering which is considered very early in the season. This is similarly the case with the pollen parent, blueberry plant ‘03-2’, and which also has a mid-season flowering date. Still further, the new variety of blueberry plant is distinguishable from its seed parent in view of the fruit size produced by the new plant. In this regard, the new variety of blueberry plant produces large to very large fruit in relative comparison to the seed parent, and which produces only medium to large fruit; and the pollen parent which produces large sized blueberries.

The present, new and novel variety of blueberry plant is distinguishable from the most closely known variety, that being blueberry plant ‘EB 8-1’ [patent pending, U.S. patent application Ser. No. 13/986,863, and which was filed on Jun. 12, 2013] in view of the differing plant vigor; displayed growth habit; fruit bloom intensity; fruit firmness; fruit acidity; fruit weight, and other plant characteristics which will be discussed in the specification which follows. Still further, the new variety is clearly distinguishable from the common commercial blueberry plant variety ‘Ridley 1111’ [unpatented]. The blueberry plant variety ‘Ridley 1111’ produces medium to large sized fruit in comparison to the new variety of blueberry parent, and which produces large to very large fruit. It should be understood that the asexual propagation of the new, present variety of blueberry plant was accomplished by means of cuttings. The observations of the new and novel plants has taken place over a number of years and the inventor has repeatedly confirmed that the novel characteristics of the present, new variety of blueberry plant are stable across multiple generations.

### BRIEF DESCRIPTION OF THE DRAWING

The accompanying drawing which is provided is a color photograph of the new blueberry plant ‘EB 9-2’ and which is juxtaposed relative to an image of the closest known variety, that being ‘EB 8-1’ [patent pending]. The photograph illustrates the new variety, and shows a portion of a vegetative stem which displays the growth habit of the leaves; one leaf is further illustrated showing the dorsal coloration thereof; and several fruit are depicted showing both the dorsal and ventral coloration, and the morphology of same. The photograph additionally shows a vegetative stem showing typical leaves and the fruit produced by the closest known variety, that being blueberry plant ‘EB 8-1’ [patent pending].

The color in this photograph is as nearly true as is possible in a color representation of this type. Due to chemical development, processing, and printing, the leaves and fruit depicted in this photograph may or may not be accurate when compared to the actual specimens. For this reason, future color references should be made to the color plates, and descriptions provided hereinafter. Common color names are also employed in the specification which follows.

### NOT A COMMERCIAL WARRANTY

The following detailed descriptions were prepared solely to comply with the provisions of 35 U.S.C. §112, and does not constitute a commercial warranty (either expressed or implied) that the present plant variety will, in the future, display the botanical, horticultural or other characteristics as set forth, hereinafter. Therefore, this disclosure may not be relied upon to support any future legal claims including, but not limited to, breach of warranty of merchantability, or fitness for any particular purpose, or non-infringement, which is directed, in whole, or in part, to the present new variety.
Plant:
   Plant vigor.—Generally considered strong to very strong. This in contrast to the closest known variety ‘EB 8-1’ [patent pending, U.S. patent application Ser. No. 13/986,863], and where this plant characteristic is merely considered medium or average.
   Plant growth habit.—Considered upright to semi-upright. This in contrast to the closest known variety ‘EB 8-1’, and which displays a semi-spreading growth habit.
   Color.—One Year Old Shoots — Green. This color is virtually indistinguishable from the same plant characteristic as expressed by the closest known variety ‘EB 8-1’.
   Internode length.—One Year Old Shoots — These are considered long to very long for the species. This in contrast to the same growth habit as expressed by the closest known variety ‘EB 8-1’, and which displays a medium to long internode length.
   Leaf length.—Considered short to average in length, about 61.13 millimeters. This in contrast to the closest known variety ‘EB 8-1’, and which has a medium to long leaf length of about 63.17 millimeters.
   Leaf width.—Considered narrow to medium in width, about 30.57 millimeters. This is in contrast to that same displayed plant characteristic of the closest known variety ‘EB 8-1’, and which is considered medium to broad in width, and which further has a displayed width, on average, of about 29.6 millimeters.
   Leaf.—Ratio of Length Versus Width — Considered large for the species. This is in contrast to that characteristic displayed by the closest known variety ‘EB 8-1’ and which is only considered medium to large.
   Leaf shape.—Considered ovate.
   Leaf color.—Dorsal surface — Green.
   Leaf color.—Intensity — The dorsal surface color intensity is considered average for the species.
   Leaf marginal edge.—Entire.
Flowers:
   Flower bud coloration.—The anthocyanin coloration is considered very weak.
   Inflorescence.—Length — Considered average for the species.
   Flower.—Corolla Shape — Urceolate.
   Flower.—Corolla Tube Size — Considered medium.
   Corolla tube coloration.—The anthocyanin coloration is considered absent or very weak.
   Corolla tube surface texture.—Ridges are present.
Fruit:
   Fruit cluster density.—Generally considered average to dense. This is in contrast to the blueberry variety ‘EB 8-1’, and where this plant displays an average fruit cluster density.
   Unripe fruit coloration.—Considered a light to medium green.
   Fruit size.—Considered large to very large for the species. This is in contrast to the closest known variety ‘EB 8-1’, and where the fruit is considered just merely large.
   Fruit shape.—Generally speaking, and when viewed in a longitudinal sectional view, the fruit shape is considered oblate.
   Fruit sepal orientation.—Generally speaking, the sepals are considered erect to semi-erect.
   Sepal type.—Generally considered reflexed. This is in contrast to the closest known variety ‘EB 8-1’, and where the same displayed plant characteristic is considered straight.
   Calyx basin diameter.—Considered average for the species, about 9.60 mm. This is in contrast to the closest known variety ‘EB 8-1’, and where the same displayed plant characteristic is considered medium to large.
   Calyx basin depth.—Considered average, about 2.97 mm.
   Intensity of fruit bloom.—Considered very strong in contrast to the closest known variety ‘EB 8-1’, and which is only considered merely strong for the variety.
   Fruit skin coloration.—Considered dark blue.
   Fruit firmness.—The fruit produced by the new variety is considered to be firm to very firm for the species. This is in contrast to the closest known variety ‘EB 8-1’, and where this displayed fruit firmness is considered merely average.
   Fruit sweetness.—Considered high for the variety. This is in contrast to the closest known variety ‘EB 8-1’, and which is considered merely average.
   Fruit acidity.—Generally — Considered low to medium for the species. This is in contrast to the closest known variety ‘EB 8-1’, and which is considered to be merely average.
   Fruiting type.—Fruiting occurs on one year old, and current season's shoots.
   Time of vegetative bud burst.—Considered very early for the species.
   Time of flowering.—One year old shoots — Generally considered very early.
   Time of flowering on current year's shoots.—Considered very early in the season for the species.
   Time of fruit ripening on one year old shoots.—Considered very early in the season for the species.
   Time of fruit ripening on current year shoots.—Considered very early in the season for the species.
   Fruit diameter.—About 21.62 millimeters, on average.
   Fruit height.—Generally about 15.43 millimeters, on average.
   Fruit weight.—About 4.79 grams, on average.
   Resistance to insects and disease.—No particular susceptibilities were noted. The present variety has not been tested to expose or detect any susceptibilities or resistances to any known plants and/or fruit diseases. Although the new variety of blueberry plants possesses the described characteristics when grown under the ecological conditions prevailing near Yanchep Springs, Western Australia, it should be understood that the variations of the usual magnitude and characteristics incident to changes in growing conditions, fertilization, pruning, pest control, frost, climatic variables and horticultural management are to be expected.

Having thus described and illustrated my new variety of blueberry plant, what I claim is new and desire to secure by plant Letters Patent is:

1. A new and distinct variety of blueberry plant, substantially as illustrated and described, and which is characterized as to novelty by producing a large to very large fruit, and which further is mature for harvesting and shipment in the very early season under the ecological conditions prevailing near Yanchep Springs, Western Australia.
